(19) United States
(12) Patent Application Publication
(10) Pub. No.: US 2025/0345232 A1
(43) Pub. Date: Nov. 13, 2025

(54) INTELLIGENT MULTIFUNCTIONAL CUPPING DEVICE FOR TRADITIONAL CHINESE MEDICINE PHYSIOTHERAPY

(71) Applicant: Guangxi Lisen Medical Device Technology Co., Ltd., Nanning (CN)

(72) Inventor: Guangwen Huang, Nanning (CN)

(21) Appl. No.: 19/266,253

(22) Filed: Jul. 11, 2025

(30) Foreign Application Priority Data
May 13, 2024 (CN) 202410586570.7

(51) Int. Cl. A61H 9/00; A61H 23/02; A61H 39/06; A61N 1/05; A61N 1/44; A61N 5/06

(52) U.S. Cl. CPC A61H 9/0057; A61H 23/02; A61H 39/06; A61N 1/0502; A61N 1/44; A61N 5/0613; A61H 2201/0157; A61H 2201/0192; A61H 2201/0207; A61H 2201/0278; A61H 2201/10; A61H 2201/1246; A61H 2201/1418; A61H 2201/5043; A61H 2201/5056; A61N 2005/0651; A61N 2005/0659

(57) ABSTRACT

An intelligent multifunctional cupping device includes a cup body and a total control upper housing; outer surface of top wall of the cup body is provided with a connection groove, the total control upper housing is connected into connection groove; a control circuit board, a vibration motor, an electric negative pressure pump, a charge/discharge battery, a negative ion generator and an air release solenoid valve are arranged on inner side of the host housing; a charging interface is provided on side wall of one end of the host housing, bottom wall of the total control upper housing is respectively provided with a negative pressure cupping air port, bioelectric current release spring needles, function release spring needles, and an infrared LED light I; the connection groove is provided with a cup body protruding cupping nozzle, bioelectric current release spring needle contact points, and host function release spring needle contact points.

### TECHNICAL FIELD

The invention relates to the technical field of physiotherapy device for traditional Chinese medicine (TCM) physiotherapy, in particular to an intelligent multifunctional cupping device for traditional Chinese medicine physiotherapy.

### BACKGROUND ART

TCM physiotherapy uses the principles of traditional Chinese medicine to perform physical therapy on the human body. Common methods include fumigation, warm moxibustion, magnetic therapy, light therapy, massage, tuina, scraping, cupping, moxibustion, acupuncture, and Bian stone therapy. These physical therapies have obvious effects on many chronic diseases caused by sub-health and have been widely promoted and applied. As the most common TCM physiotherapy method, cupping has been more widely promoted and applied.

In recent years, many multifunctional cupping cups have been promoted and applied in the TCM physiotherapy industry. The “multifunctional long-shaped cupping cup” described in Chinese Utility Model patent application No. 201922400729.9 and the “improved multifunctional cupping cup” described in patent application No. 202122079592.9 are the most representative. While the above-mentioned multifunctional cupping cups have many advantages, they also have some limitations. When performing cupping, in addition to the multifunctional cupping cup itself, a host computer for controlling the work of the multifunctional cupping cup is also required. The host computer includes devices such as an air pump, a power supply, and a control circuit board. a power interface and an air tank interface of the host computer are respectively connected to a power interface and an air tank interface of the multifunctional cupping cup, so that the host computer may control the work of the multifunctional cupping cup. Although the above-mentioned multifunctional cupping cups are widely used in professional physiotherapy places such as physiotherapy halls, due to the large number of accessories, physiotherapists need a large storage box to store the multifunctional cupping cups and the host computer when going out to provide on-site physiotherapy services to customers, which is inconvenient to carry. It is also inconvenient for outdoor work or tourists to carry, and its use range is limited due to its large volume and inconvenient carrying. The need for an external host computer, and the repeated plugging and unplugging of the power supply and air tank during use will also affect the service life of the multifunctional cupping cup and the host computer. In addition, the host computer and the multifunctional cupping cup are produced separately, resulting in low production efficiency, high cost, difficult improvement of output, and difficulty in meeting the needs of the majority of customers.

Existing other multifunctional physiotherapy devices also have more than 10 functions, which are distributed on 4-5 probes. For example, when performing physiotherapy on multiple parts of the human body, each physiotherapy part is operated by 4-5 probes respectively. Operators need to perform tedious operations and spend a lot of working time, resulting in very low work efficiency. They also need to hold the physiotherapy device in their hands throughout the operation, resulting in high labor intensity and high labor costs. At the same time, since not all functional physiotherapies are carried out simultaneously, the non-cooperative treatment between functions affects the curative effect more.

### SUMMARY OF THE INVENTION

The invention aims at the above shortcomings and provide an intelligent multifunctional cupping device, which can work independently, has a small volume and is convenient to carry, has multiple functions and good physiotherapy effects, and is conducive to popularization and application.

The technical solution adopted by the invention to achieve the above objectives is as follows: an intelligent multifunctional cupping device includes a cup body and a total control upper housing connected to the cup body; the cup body includes a cup mouth and a cup wall extending upward from the cup mouth, an outer surface of a top wall of the cup body is provided with a downwardly recessed connection groove; the total control upper housing is connected into the connection groove; the total control upper housing includes a host housing; a control circuit board and a vibration motor are arranged on an inner side of the host housing. An electric negative pressure pump, a charge/discharge battery, a negative ion generator and an air release solenoid valve are also arranged on the inner side of the host housing; a charging interface is provided on a side wall of one end of the host housing, the charging interface is connected to the charge/discharge battery through a circuit; a bottom wall of the total control upper housing is respectively provided with a negative pressure cupping air port, bioelectric current release spring needles, function release spring needles, and an infrared LED light I; the connection groove on the top wall of the cup body is provided with a cup body protruding cupping nozzle, bioelectric current release spring needle contact points, and host function release spring needle contact points; an upper end of the cup body protruding cupping nozzle in the connection groove is docked with the negative pressure cupping air port on the bottom wall of the total control upper housing, a lower end of the cup body protruding cupping nozzle is connected with a negative pressure cupping air inlet, the negative pressure cupping air inlet extends downward through a bottom wall of the connection groove and communicates with the cup body inner cavity; an air pump inlet/outlet is provided on one side wall of the host housing, the air pump inlet/outlet is connected with one air nozzle of the electric negative pressure pump and the air release solenoid valve through air pipes; another air nozzle of the electric negative pressure pump is connected with the air release solenoid valve and the negative pressure cupping air port through air pipes; microcurrent conductive silicone strips are connected to inner surface of a cup mouth, the bioelectric current release spring needle contact points are connected with the microcurrent conductive silicone strips through circuits.

As a further technical solution of the invention, the control circuit board is respectively connected to the charge/discharge battery, vibration motor, electric negative pressure pump, negative ion generator, air release solenoid valve, bioelectric current release spring needles, function release spring needles, and infrared LED light I; the bioelectric current release spring needles on the bottom wall of the total control upper housing are docked with the bioelectric current release spring needle contact points in the connection groove, and the function release spring needles on the bottom wall of the total control upper housing is docked with the host function release spring needle contact points in the connection groove, a heating plate, a quantum generation plate, a negative ion release head, and a terahertz chip are arranged on a top wall of the cup body inner cavity below the connection groove; the host function release spring needle contact points are respectively connected with the heating plate, the quantum generation plate, the negative ion release head, and the terahertz chip through conductive lines, a lower side of the negative ion release head is provided with a negative ion release port penetrating the cup body inner cavity.

As a further technical solution of the invention, a terahertz stone is also arranged on the top wall of the cup body inner cavity below the connection groove, the terahertz stone is set below a heating plate I.

As a further technical solution of the invention, downwardly recessed lateral installation grooves are respectively arranged on both sides of the connection groove, an LED light mounting bracket is protrudingly arranged in the lateral installation groove, and an infrared LED light II is installed on the LED light mounting bracket, a magnet I, a heating plate III, and a Bian stone I are also arranged on a bottom wall of the lateral installation groove, the magnet I and the heating plate III are respectively connected with the host function release spring needle contact points in the connection groove through connection circuits, the Bian stone I is located directly below the heating plate III, a cover plate for closing the lateral installation groove is arranged on an upper side of the lateral installation groove.

As a further technical solution of the invention, a magnetic combined cup inner spring silicone release function combination head that can be adsorbed under a top wall of a cup body inner cavity is also arranged in the cup body inner cavity, and the magnetic combined cup inner spring silicone release function combination head includes a combination head body surrounded by a telescopic spring silicone; the length, width, and height dimensions of the combination head body are respectively smaller than those of the cup body inner cavity; a bottom end and a top end of the combination head body are respectively provided with a bottom sealing cover plate and a top sealing cover plate for closing an inner cavity of the combination head body; a telescopic silicone inner vibration motor, and a heating plate II, and an infrared LED light III are arranged in the inner cavity of the combination head body; a transition power and function spring needle group are also arranged on the bottom wall of the connection groove of the cup, a magnetic switch is also arranged on the bottom wall of the connection groove on one side of the transition power and function spring needle group, the top sealing cover plate is provided with a transition power and function spring needle group connector docked with the transition power and function spring needle group, and the transition power and function spring needle group connector are respectively connected with the telescopic silicone inner vibration motor and the heating plate II in the inner cavity of the combination head body through conductive lines; magnets II are arranged in the top sealing cover plate, and magnets I corresponding to the magnets II in position one by one are arranged on the top wall of the cup body inner cavity.

As a further technical solution of the invention, the bottom sealing cover plate is provided with a heating plate installation position, a magnet installation position, and a Bian stone installation position located below the heating plate installation position; the heating plate II is arranged in the heating plate installation position, the magnet III is arranged in the magnet installation position, the magnet III is connected with the transition power and function spring needle group connector through conductive lines, a Bian stone II is arranged in the Bian stone installation position.

As a further technical solution of the invention, a bottom end of the host housing is provided with a protrusion that is configured positioned in the connection groove of the top wall of the cup body, the negative pressure cupping air port, the bioelectric current release spring needles, the function release spring needles, and the infrared LED light I are respectively arranged in a bottom wall of the protrusion; the bottom wall of the total control upper housing on both sides of the protrusion is set as an arc-shaped groove structure that cooperates with an arc-shaped surface of a top of the cup wall, the air pump inlet/outlet is arranged in the arc-shaped groove on one side of the protrusion and penetrates to an inner cavity of the total control upper housing.

As a further technical solution of the invention, the cup body can be a long-shaped cup, a chest cup, hip cup, or abdominal cup.

As a further technical solution of the invention, connection magnets II are respectively arranged at both ends of the connection groove, and connection magnets I corresponding to the connection magnets II in position are provided on both sides of a bottom end of the total control upper housing, the connection magnets II and the connection magnets I attract each other.

As a further technical solution of the invention, a top surface of the total control upper housing is respectively provided with a digital display screen for device function intensity, a power indicator light, and function control buttons, which are respectively connected with the control circuit board in the total control upper housing through circuits.

The intelligent multifunctional cupping device of the invention has the following beneficial effects:
1. the total control upper housing is equipped with a control circuit board, vibration motor, electric negative pressure pump, charge/discharge battery, negative ion generator, and air release solenoid valve. The bottom wall of the total control upper housing is provided with a negative pressure cupping air port, bioelectric current release spring needles, function release spring needles, and infrared LED lights; the connection groove on a top wall of the can body is equipped with a cup body protruding cupping nozzle, bioelectric current release spring needle contact points, and host function release spring needle contact points. By installing the total control upper housing in the connection groove of the can body's top wall, cupping and related TCM physiotherapy can be performed without external hosts or power supplies. The cupping device is compact and portable, suitable for both commercial use and home and travel use;
2. the invention designs the total control upper housing and the cup body separately, the same connection groove is set on top walls of different can bodies, which can be designed in different lengths and sizes (such as chest cups, hip cups, or abdominal cups, etc.) to meet the conditioning needs of the whole body. It can be used for cupping and physiotherapy on different parts, from the neck, head, and feet (with the smallest size) to the chest and abdomen, realizing multi-functional use of one device and reducing the investment cost of TCM physiotherapy equipment;
3. the total control upper housing is equipped with a control circuit board and an air release solenoid valve, which can automatically adjust the air pressure in the cup. During cupping, the operator only needs to adjust the appropriate gear, eliminating the need to learn complicated operations. Both children and the elderly can quickly master the use, and practitioners can quickly grasp TCM meridian physiotherapy, encouraging TCM enthusiasts to learn through successful physiotherapy and patient recognition, and boosting their confidence in engaging in TCM physiotherapy;
4. for thin or muscular individuals, the skin is difficult to be sucked to the top of the can during cupping, affecting the physiotherapy effect. The invention sets a magnetic combined cup inner spring silicone release function combination head in the cup body inner cavity, which can be adsorbed under the top wall of the cup body inner cavity. In the magnetic combined cup inner spring silicone release function combination head is equipped with a telescopic silicone vibration motor, heating plate II, magnet III, and Bian stone II. When used by thin or muscular individuals, installing the magnetic combined cup inner spring silicone release function combination head into the cup makes it easier for the skin to be sucked to a bottom of the magnetic combined cup inner spring silicone release function combination head during cupping, achieving a more ideal physiotherapy effect;
5. the invention enables simultaneous multiple TCM physiotherapies during cupping, including massage, warm moxibustion, magnetic therapy, infrared rays, microcurrent, terahertz therapy, negative ions, Bian stone therapy, and quantum cupping therapy. The simultaneous implementation of multiple therapies enhances the therapeutic effect, significantly improves the efficiency of TCM meridian physiotherapy, shortens treatment time, reduces patient pain and costs, improves immunity, reduces illness, alleviates hospital resource burdens, enhances the understanding of TCM physiotherapy, and promotes TCM culture, achieving the benefits of both disease treatment and preventive health care.

The intelligent multifunctional cupping device of the invention is further described below in conjunction with the accompanying drawings and embodiments.

### BRIEF DESCRIPTION OF ACCOMPANY DRAWINGS

FIG. 1 is an exploded view of an intelligent multifunctional cupping device of the invention (with a partial cross-section of a total control upper housing).

FIG. 2 is an assembly diagram of an intelligent multifunctional cupping device shown in FIG. 1.

FIG. 3 is a structural schematic diagram (partial cross-section) of the cup body in Embodiment 2 of the intelligent multifunctional cupping device of the invention.

Explanation of reference numerals: 1—infrared LED light II, 2—magnet I, 3—Bian stone I, 4—cup body, 5—heating plate III, 6—air pump inlet/outlet, 7—vibration motor, 8—electric negative pressure pump, 9—charging interface, 10—host housing, 11—power indicator light, 12—function control button, 13—digital display screen for device function intensity, 14—control circuit board, 15—negative ion generator, 16—air release solenoid valve, 17—charge/discharge battery, 18—connection magnet I, 19—function release spring needle, 20—bioelectric current release spring needle, 21—infrared LED light I, 22—negative pressure cupping air port, 23—connection magnet II, 24—cup body protruding cupping nozzle, 25—bioelectric current release spring needle contact point, 26—host function release spring needle contact point, 27—connection groove, 28—negative ion release head, 29—negative ion release port, 30—quantum generation plate, 31—terahertz chip, 32—terahertz stone, 33—negative pressure cupping air inlet, 34—microcurrent conductive silicone strip, 35—cup body inner cavity, 36—cup mouth, 37—cup wall, 38—LED light mounting bracket, 39—protrusion, 40—heating plate I, 41—transition power and function spring needle group connector, 42—transition power and function spring needle group, 43—magnetic switch, 44—infrared LED light III, 45—magnet III, 46—telescopic silicone inner vibration motor, 47—Bian stone II, 48—heating plate II, 49—bottom sealing cover plate, 50—magnetic combined cup inner spring silicone release function combination head, 51—combination head body, 52—top sealing cover plate, 53—magnet II.

### SPECIFIC EMBODIMENT OF THE INVENTION

### Embodiment 1

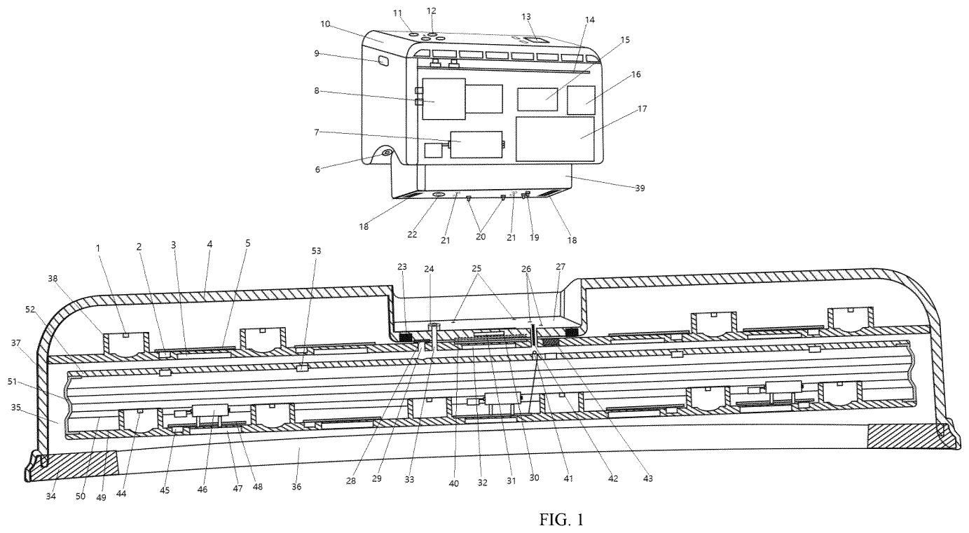

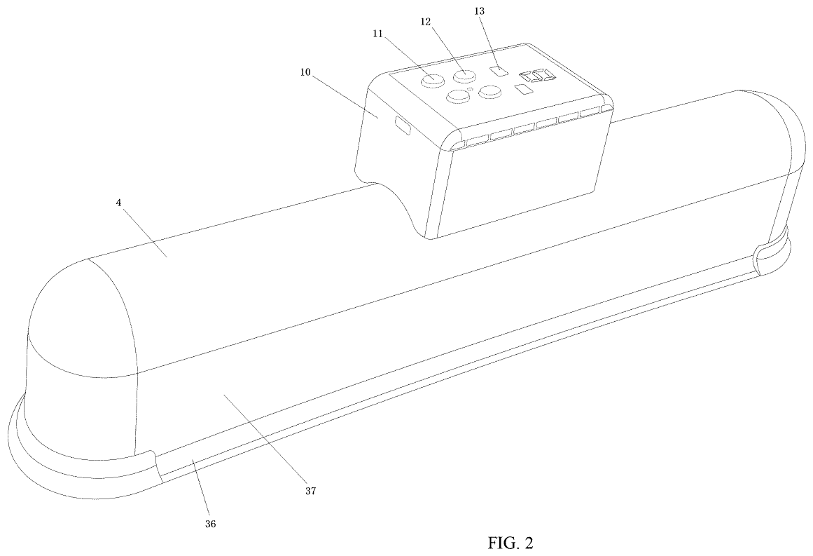

As shown in FIG. 1 and FIG. 2, an intelligent multifunctional cupping device of the invention includes a cup body 4 and a total control upper housing connected to the cup body 4. In this embodiment, the cup body 4 is a long-shaped cup, and the size of the long-shaped cup is not limited to the size in the drawings; the long-shaped cup body is a cup with a length of 6-40 cm. Of course, the cup body 4 can also be a chest cup, hip cup, or abdominal cup, which is made of transparent or translucent materials. The cup body 4 includes a cup mouth 36 and a cup wall 37 extending upward from the cup mouth 36, an outer surface of a top wall of the cup body 4 is provided with a downwardly recessed connection groove 27. During use, the total control upper housing is connected to the connection groove 27 at a top end of the cup body 4. Connection magnets II 23 are respectively arranged at both ends of the connection groove 27, and connection magnets I 18 corresponding to the connection magnets II 23 in position are provided on both sides of a bottom end of the total control upper housing. When the total control upper housing is connected in the connection groove 27, the connection magnets II 23 and the connection magnets I 18 attract each other.

As shown in FIG. 1 and FIG. 2, the total control upper housing includes a host housing 10, and a control circuit board 14, a vibration motor 7, an electric negative pressure pump 8, a charge/discharge battery 17, a negative ion generator 15, and an air release solenoid valve 16 are arranged inside the host housing 10; the control circuit board 14 is a main board of the intelligent multifunctional cupping device, one end of the vibration motor 7 is connected with an eccentric cam, and the eccentric cam rotates to generate vibration when the vibration motor 7 works; a charging interface 9 is provided on a side wall of one end of the host housing 10; the charging interface 9 is a 5-volt Type-C charging port, which is connected to the charge/discharge battery 17 through a circuit, and the charge/discharge battery 17 is charged through the charging interface 9; a bottom wall of the host housing 10 of the total control upper housing is respectively provided with a negative pressure cupping air port 22, bioelectric current release spring needles 20, function release spring needles 19, and an infrared LED light I 21; the bioelectric current release spring needles 20 and the function release spring needles 19 are conductive terminals embedded in a bottom wall of the total control upper housing with the bottom ends protruding from a bottom surface of bottom wall. The infrared LED light I 21 is also embedded in the bottom wall of the total control upper housing. Since both the bottom wall of the total control upper housing and a top wall of the cup body 4 are made of transparent or translucent materials, the infrared LED light I 21 irradiate surface of the human skin in a cup body inner cavity 35 through the bottom wall of the total control upper housing and the top wall of the cup body 4. The control circuit board 14 is respectively connected with the charge/discharge battery 17, the vibration motor 7, the electric negative pressure pump 8, the negative ion generator 15, the air release solenoid valve 16, the bioelectric current release spring needle 20, the function release spring needles 19, and the infrared LED light I 21 through circuits; a top surface of the total control upper housing is respectively provided with a digital display screen for device function intensity 13, a power indicator light 11, and function control buttons 12, which are respectively connected with the control circuit board 14 in the total control upper housing through circuits. An air pump inlet/outlet 6 is provided on one side wall of the host housing 10, which is connected with one air nozzle of the electric negative pressure pump 8 and the air release solenoid valve 16 through air pipes; an other air nozzle of the electric negative pressure pump 8 is connected with the air release solenoid valve 16 and the negative pressure cupping air port 22 through air pipes; the air intake and exhaust of the electric negative pressure pump 8 both pass through the air pump inlet/outlet 6, and the air release solenoid valve 16 is used to adjust the air pressure in the cup body inner cavity 35. During the cupping process, the air outlet and inlet of the cup body all pass through the air pump inlet/outlet 6. The role of the air release solenoid valve 16 is to adjust the air pressure in the cup. When the air pressure is high, the electric negative pressure pump 8 is controlled to release air, and when the air pressure is low, the electric negative pressure pump 8 is controlled to inhale air, so as to achieve the purpose of automatically adjusting the air pressure. In the embodiment, a bottom end of the host housing 10 is provided with a protrusion 39 that is configured positioned in the connection groove 27 of the top wall of the cup body 4. The negative pressure cupping air port 22, the bioelectric current release spring needles 20, the function release spring needles 19, and the infrared LED light I 21 are respectively arranged in a bottom wall of the protrusion 39. The bottom wall of the total control upper housing on both sides of the protrusion 39 is set as an arc-shaped groove structure that cooperates with an arc-shaped surface of a top of the cup wall 37. The air pump inlet/outlet 6 is arranged in the arc-shaped groove on one side of the protrusion 39 and penetrates to an inner cavity of the total control upper housing.

As shown in FIG. 1 and FIG. 2, the connection groove 27 on the top wall of the cup body 4 is provided with a cup body protruding cupping nozzle 24, bioelectric current release spring needle contact points 25, and host function release spring needle contact points 26; the bioelectric current release spring needle contact points 25 and the host function release spring needle contact points 26 are conductive terminals embedded in the top wall of the cup body, a lower end of the cup body protruding cupping nozzle 24 is connected with a negative pressure cupping air inlet 33, which extends downward through a bottom wall of the connection groove 27 and communicates with the cup body inner cavity 35. When the total control upper housing is connected in the connection groove 27, an upper end of the cup body protruding cupping nozzle 24 is docked with the negative pressure cupping air port 22 on the bottom wall of the total control upper housing, and the electric negative pressure pump 8 sucks air through an air pipe connected to the negative pressure cupping air port 22, the cup body protruding cupping nozzle 24, and the negative pressure cupping air inlet 33 for cupping. The bioelectric current release spring needles 20 on the bottom wall of the total control upper housing are docked with the bioelectric current release spring needle contact points 25 in the connection groove 27, and this route is used to transmit microcurrents. The function release spring needles 19 on the bottom wall of the total control upper housing is docked with the host function release spring needle contact points 26 in the connection groove 27, and this route is used to transmit currents of other functions.

As shown in FIG. 1, a heating plate, a quantum generation plate 30, a negative ion release head 28, and a terahertz chip 31 are arranged on a top wall of the cup body inner cavity 35 below the connection groove 27. Corresponding installation positions are set on the top wall of the cup body inner cavity 35 below the connection groove 27, and the heating plate, the quantum generation plate 30, the negative ion release head 28, and the terahertz chip 31 are respectively installed in the corresponding installation positions. The quantum generation plate 30 cup generate quanta after being powered on, the negative ion generator 15 cup generate negative ions when working, and the terahertz chip 31 cup generate terahertz waves and infrared rays when heated, which cup activate water and produce a resonance cell penetration effect, having a good effect on physiotherapy. The host function release spring needle contact points 26 are respectively connected with the heating plate, the quantum generation plate 30, the negative ion release head 28, and the terahertz chip 31 through conductive lines, which are embedded in the top wall of the cup body inner cavity 35 below the connection groove 27. One end of the conductive line is connected with the host function release spring needle contact points 26, and the other end of the conductive line extends to the corresponding installation position. A lower side of the negative ion release head 28 is provided with a negative ion release port 29 penetrating the cup body inner cavity 35. During the physiotherapy process, negative ions enter the cup body inner cavity 35 through the negative ion release port 29 via the negative ion release head 28 and contact the human skin; microcurrent conductive silicone strips 34 are connected to inner surface of a cup mouth 36, which are symmetrically arranged on opposite side surfaces inside the cup mouth 36. The bioelectric current release spring needle contact points 25 are connected with the microcurrent conductive silicone strips 34 through circuits. A terahertz stone 32 is also arranged on the top wall of the cup body inner cavity 35 below the connection groove 27. An installation position for the terahertz stone 32 is set below a heating plate I 40 on the top wall of the cup body inner cavity 35 below the connection groove 27. The terahertz stone 32 is installed and fixed in the installation position. After the heating plate I 40 is heated, the terahertz stone is warmed by moxibustion, which has the effects of promoting blood circulation, improving sleep quality, relieving fatigue, improving immunity, and promoting skin repair.

As shown in FIG. 1, downwardly recessed lateral installation grooves are respectively arranged on both sides of the connection groove 27. An LED light mounting bracket 38 is protrudingly arranged in the lateral installation groove, and an infrared LED light II 1 is installed on the LED light mounting bracket 38. A magnet I 2, a heating plate III 5, and a Bian stone I 3 are also arranged on a bottom wall of the lateral installation groove. An installation position is set on the bottom wall of the lateral installation groove, and the magnet I 2 and the heating plate III 5 are respectively installed in the installation position. The Bian stone I 3 is located directly below the heating plate III 5, the magnet I 2 and the heating plate III 5 are respectively connected with the host function release spring needle contact points 26 in the connection groove 27 through connection circuits (not shown in the drawings), and the circuits is configured to be embedded in the bottom wall of the lateral installation groove. A cover plate for closing the lateral installation groove is arranged on an upper side of the lateral installation groove. When the heating plate III 5 is energized and heated, the Bian stone I 3 is warmed by moxibustion, and the warmed Bian stone has the effects of promoting blood circulation and qi, and dredging the meridians.

In this embodiment, a magnetic combined cup inner spring silicone release function combination head 50 that can be adsorbed under a top wall of a cup body inner cavity is also arranged in the cup body inner cavity. The magnetic combined cup inner spring silicone release function combination head 50 includes a combination head body 51 surrounded by a telescopic spring silicone. The length, width, and height dimensions of the combination head body 51 are respectively smaller than those of the cup body inner cavity. When the magnetic combined cup inner spring silicone release function combination head 50 is installed in the cup body inner cavity, there are still some gaps between the inner wall of the cup and the magnetic combined cup inner spring silicone release function combination head 50; a bottom end and a top end of the combination head body 51 are respectively provided with a bottom sealing cover plate 49 and a top sealing cover plate 52 for closing an inner cavity of the combination head body 51, which are both plastic plates. A telescopic silicone inner vibration motor 46, a heating plate II 48, and an infrared LED light III 44 are arranged in the inner cavity of the combination head body 51. A transition power and function spring needle group 42 are also arranged on the bottom wall of the connection groove of the cup, which is a spring needle group (which is configure as multiple conductive terminals embedded in the bottom wall of the connection groove of the cup) for supplying power to components in the inner cavity of the combination head body 51 and adjusting functions. The transition power and function spring needle group 42 is conductively connected with the host function release spring needle contact points 26; a magnetic switch 43 is also arranged on the bottom wall of the connection groove on one side of the transition power and function spring needle group 42. When the magnetic combined cup inner spring silicone release function combination head 50 is installed in the cup body inner cavity and touches the magnetic switch 43, a power supply of the heating plate, infrared LED light, and magnet on the cup is disconnected, and only components in the inner cavity of the combination head body 51 are energized at this time. After the magnetic combined cup inner spring silicone release function combination head 50 is removed from the cup, the power supply of the heating plate, infrared LED light, and magnet in the cup is turned on, and surface of the magnetic combined cup inner spring silicone release function combination head 50 can be cleaned at this time. The bottom sealing cover plate 49 is provided with a heating plate installation position, a magnet installation position, and a Bian stone installation position located below the heating plate installation position; the heating plate II 48 is arranged in the heating plate installation position, the magnet III 45 is arranged in the magnet installation position, the infrared LED light III 44 is installed on the LED light mounting bracket inside the bottom sealing cover plate 49, the infrared LED light III 44 and the magnet III 45 are respectively connected with the transition power and function spring needle group connector 41 through conductive lines, a Bian stone II 47 is arranged in the Bian stone installation position, a bottom surface of the Bian stone II 47 protrudes from a bottom surface of the bottom sealing cover plate 49, the heating plate II 48 is energized and heated to warm the Bian stone II 47 by moxibustion, and the warmed Bian stone has the effects of promoting blood circulation and qi, and dredging the meridians. The top sealing cover plate 52 is provided with a transition power and function spring needle group connector 41 docked with the transition power and function spring needle group 42, and the transition power and function spring needle group connector 41 are respectively connected with the telescopic silicone inner vibration motor 46 and the heating plate II 48 in the inner cavity of the combination head body 51 through conductive lines; magnets II 53 are arranged in the top sealing cover plate 52, and magnets I 2 corresponding to the magnets II 53 in position one by one are arranged on the top wall of the cup body inner cavity. When the magnetic combined cup inner spring silicone release function combination head 50 is installed in the cup body inner cavity, the magnet II 53 in the top sealing cover plate 52 and the magnets I 2 on a top wall of the cup body inner cavity are mutually adsorbed and connected to form a whole.

Before use, the charge/discharge battery 17 in the total control upper housing is fully charged through the charging interface 9. During use, the total control upper housing is connected in the connection groove 27 at the top end of the cup body, and the connection magnets II 23 in the connection groove 27 of the cup body and the connection magnets I 18 on both sides of the bottom end of the total control upper housing are mutually adsorbed. The negative pressure cupping air port 22, the cup body protruding cupping nozzle 24, and the negative pressure cupping air inlet 33 are sequentially connected, and the electric negative pressure pump 8 is connected to the negative pressure cupping air port 22, the cup body protruding cupping nozzle 24, and the negative pressure cupping air inlet 33 through air pipes to pump air for cupping. The bioelectric current release spring needles 20 are docked with the bioelectric current release spring needle contact points 25, and the function release spring needles 19 are docked with the host function release spring needle contact points 26. Through the function control buttons 12 on the top surface of the total control upper housing, a variety of composite TCM physiotherapies cup be performed on the physiotherapy recipient, including cupping, massage, warm moxibustion, magnetic therapy, infrared ray, microcurrent, terahertz therapy, negative ion, quantum, etc.

When thin and muscular people receive physiotherapy, the skin of thin and muscular people is difficult to be sucked to the top of the cup during the cupping physiotherapy process, which will affect the physiotherapy effect. At this time, the magnetic combined cup inner spring silicone release function combination head 50 is installed in the cup body inner cavity, the magnetic combined cup inner spring silicone release function combination head 50 touches the magnetic switch 43, the power supply of the heating plate, infrared LED light and magnet in the cup is disconnected, and only components in the inner cavity of the combination head body 51 are energized at this time. Physiotherapy cup be performed on thin and muscular people, and the skin of the physiotherapy recipient cup be sucked to a bottom of the magnetic combined cup inner spring silicone release function combination head 50 or contact the bottom of the magnetic combined cup inner spring silicone release function combination head 50 during the physiotherapy process, so as to achieve a more ideal physiotherapy effect.

### Embodiment 2

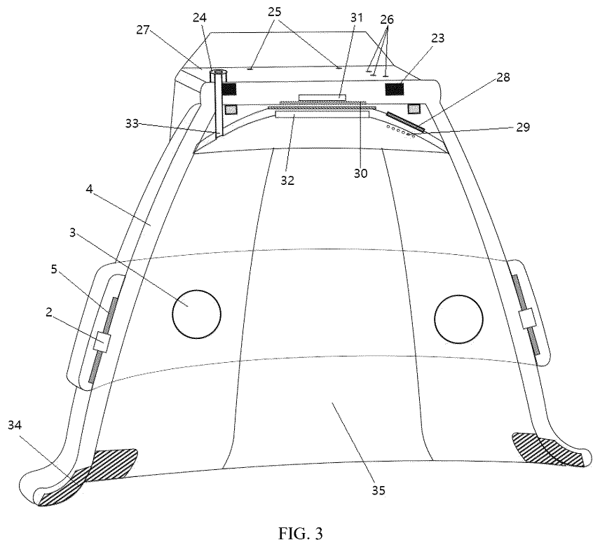

As shown in FIG. 3, an intelligent multifunctional cupping device of the invention includes a cup body and a total control upper housing connected to the cup body. In this embodiment, the cup body is a chest cup, and the structure of the total control upper housing is completely the same as that in Embodiment 1, so the total control upper housing is no longer described here. The cup body includes a cup mouth 36 and a cup wall 37 extending upward from the cup mouth 36, and an outer surface of a top wall of the cup body is provided with a downwardly recessed connection groove 27.

As shown in FIG. 3, the connection groove 27 on the top wall of the cup body is provided with a cup body protruding cupping nozzle 24, bioelectric current release spring needle contact points 25, and host function release spring needle contact points 26; the bioelectric current release spring needle contact points 25 and the host function release spring needle contact points 26 are conductive terminals embedded in the top wall of the cup body, a lower end of the cup body protruding cupping nozzle 24 is connected with a negative pressure cupping air inlet 33, which extends downward through a bottom wall of the connection groove 27 and communicates with a cup body inner cavity 35. When the total control upper housing is connected in the connection groove 27, an upper end of the cup body protruding cupping nozzle 24 is docked with the negative pressure cupping air port 22 on a bottom wall of the total control upper housing, and the electric negative pressure pump 8 is connected to the negative pressure cupping air port 22, the cup body protruding cupping nozzle 24, and the negative pressure cupping air inlet 33 through air pipes to pump air for cupping. A bioelectric current release spring needle 20 on the bottom wall of the total control upper housing is docked with the bioelectric current release spring needle contact point 25 in the connection groove 27, and this route is used to transmit microcurrents. The function release spring needles 19 on the bottom wall of the total control upper housing is docked with the host function release spring needle contact points 26 in the connection groove 27, and this route is used to transmit currents of other functions.

As shown in FIG. 3, a heating plate, a quantum generation plate 30, a negative ion release head 28, and a terahertz chip 31 are arranged on a top wall of the cup body inner cavity 35 below the connection groove 27, and corresponding installation positions are set on the top wall of the cup body inner cavity 35 below the connection groove 27; the heating plate, the quantum generation plate 30, the negative ion release head 28, and the terahertz chip 31 are respectively installed in the corresponding installation positions; the host function release spring needle contact points 26 are respectively connected with the heating plate, the quantum generation plate 30, the negative ion release head 28, and the terahertz chip 31 through conductive lines, which are embedded in the top wall of the cup body inner cavity 35 below the connection groove 27. One end of the conductive line is connected with the host function release spring needle contact point 26, and an other end of the conductive line extends to the corresponding installation position; a lower side of the negative ion release head 28 is provided with a negative ion release port 29 penetrating the cup body inner cavity 35. During the physiotherapy process, negative ions enter the cup body inner cavity 35 through the negative ion release port 29 via the negative ion release head 28 and contact the human skin. A microcurrent conductive silicone strip 34 is connected to an inner surface of the cup mouth 36, which is symmetrically arranged on opposite side surfaces inside the cup mouth 36. The bioelectric current release spring needle contact point 25 is connected with the microcurrent conductive silicone strip 34 through a circuit. A terahertz stone 32 and a Bian stone I 3 are also arranged on the top wall of the cup body inner cavity 35 below the connection groove 27, and installation positions for the terahertz stone 32 and the Bian stone I 3 are set on the top wall of the cup body inner cavity 35 below the connection groove 27. The terahertz stone 32 and the Bian stone I 3 are installed and fixed in the installation positions. Lateral installation grooves are respectively arranged on both sides of the cup wall 37. An infrared LED light II 1 is arranged in the lateral installation groove, a heating plate is also arranged inside the lateral installation groove, the heating plate is respectively connected with the host function release spring needle contact points 26 in the connection groove 27 through connection circuits, the circuit is embedded inside the cup wall, and a cover plate for closing the lateral installation groove is arranged outside the lateral installation groove. The use is the same as that in Embodiment 1 and is no longer described herein.

The above embodiments are only preferred embodiments of the invention, and the structure of the invention is not limited to the forms listed in the above embodiments. Any modifications, equivalent substitutions, etc. made within the spirit and principles of the invention shall be included in the protection scope of the invention.

1. An intelligent multifunctional cupping device, including a cup body and a total control upper housing connected to the cup body; the cup body includes a cup mouth and a cup wall extending upward from the cup mouth, an outer surface of a top wall of the cup body is provided with a downwardly recessed connection groove; the total control upper housing is connected into the connection groove; the total control upper housing includes a host housing; a control circuit board and a vibration motor are arranged on an inner side of the host housing; an electric negative pressure pump, a charge/discharge battery, a negative ion generator and an air release solenoid valve are also arranged on the inner side of the host housing; a charging interface is provided on a side wall of one end of the host housing, the charging interface is connected to the charge/discharge battery through a circuit; a bottom wall of the total control upper housing is respectively provided with a negative pressure cupping air port, bioelectric current release spring needles, function release spring needles, and an infrared LED light I; the connection groove on the top wall of the cup body is provided with a cup body protruding cupping nozzle, bioelectric current release spring needle contact points, and host function release spring needle contact points; an upper end of the cup body protruding cupping nozzle in the connection groove is docked with the negative pressure cupping air port on the bottom wall of the total control upper housing, a lower end of the cup body protruding cupping nozzle is connected with a negative pressure cupping air inlet, the negative pressure cupping air inlet extends downward through a bottom wall of the connection groove and communicates with a cup body inner cavity; an air pump inlet/outlet is provided on one side wall of the host housing, the air pump inlet/outlet is connected with one air nozzle of the electric negative pressure pump and the air release solenoid valve through air pipes; an other air nozzle of the electric negative pressure pump is connected with the air release solenoid valve and the negative pressure cupping air port through air pipes; microcurrent conductive silicone strips are connected to inner surface of a cup mouth, the bioelectric current release spring needle contact points are connected with the microcurrent conductive silicone strips through circuits.

2. The intelligent multifunctional cupping device according to claim 1, wherein the control circuit board is respectively connected to the charge/discharge battery, vibration motor, electric negative pressure pump, negative ion generator, air release solenoid valve, bioelectric current release spring needles, function release spring needles, and infrared LED light I; the bioelectric current release spring needles on the bottom wall of the total control upper housing are docked with the bioelectric current release spring needle contact points in the connection groove, and the function release spring needles on the bottom wall of the total control upper housing is docked with the host function release spring needle contact points in the connection groove, a heating plate, a quantum generation plate, a negative ion release head, and a terahertz chip are arranged on a top wall of the cup body inner cavity below the connection groove; the host function release spring needle contact points are respectively connected with the heating plate, the quantum generation plate, the negative ion release head, and the terahertz chip through conductive lines, a lower side of the negative ion release head is provided with a negative ion release port penetrating the cup body inner cavity.

3. The intelligent multifunctional cupping device according to claim 2, wherein a terahertz stone is also arranged on the top wall of the cup body inner cavity below the connection groove, the terahertz stone is set below a heating plate I.

4. The intelligent multifunctional cupping device according to claim 1, wherein downwardly recessed lateral installation grooves are respectively arranged on both sides of the connection groove, an LED light mounting bracket is protrudingly arranged in the lateral installation groove, and an infrared LED light II is installed on the LED light mounting bracket, a magnet I, a heating plate III, and a Bian stone I are also arranged on a bottom wall of the lateral installation groove, the magnet I and the heating plate III are respectively connected with the host function release spring needle contact points in the connection groove through connection circuits, the Bian stone I is located directly below the heating plate III, a cover plate for closing the lateral installation groove is arranged on an upper side of the lateral installation groove.

5. The intelligent multifunctional cupping device according to claim 1, wherein a magnetic combined cup inner spring silicone release function combination head that can be adsorbed under a top wall of the cup body inner cavity is also arranged in the cup body inner cavity, and the magnetic combined cup inner spring silicone release function combination head includes a combination head body surrounded by a telescopic spring silicone; the length, width, and height dimensions of the combination head body are respectively smaller than those of the cup body inner cavity; a bottom end and a top end of the combination head body are respectively provided with a bottom sealing cover plate and a top sealing cover plate for closing an inner cavity of the combination head body; a telescopic silicone inner vibration motor, and a heating plate II are arranged in the inner cavity of the combination head body; a transition power and function spring needle group are also arranged on the bottom wall of the connection groove of the cup, a magnetic switch is also arranged on the bottom wall of the connection groove on one side of the transition power and function spring needle group, the top sealing cover plate is provided with a transition power and function spring needle group connector docked with the transition power and function spring needle group, and the transition power and function spring needle group connector are respectively connected with the telescopic silicone inner vibration motor and the heating plate II in the inner cavity of the combination head body through conductive lines; magnets II are arranged in the top sealing cover plate, and magnets I corresponding to the magnets II in position one by one are arranged on the top wall of the cup body inner cavity.

6. The intelligent multifunctional cupping device according to claim 5, wherein the bottom sealing cover plate is provided with a heating plate installation position, a magnet installation position, and a Bian stone installation position located below the heating plate installation position; the heating plate II is arranged in the heating plate installation position, the magnet III is arranged in the magnet installation position, the magnet III is connected with the transition power and function spring needle group connector through conductive lines, a Bian stone II is arranged in the Bian stone installation position.

7. The intelligent multifunctional cupping device according to claim 1, wherein a bottom end of the host housing is provided with a protrusion that is configured positioned in the connection groove of the top wall of the cup body, the negative pressure cupping air port, the bioelectric current release spring needles, the function release spring needles, and the infrared LED light I are respectively arranged in a bottom wall of the protrusion; the bottom wall of the total control upper housing on both sides of the protrusion is set as an arc-shaped groove structure that cooperates with an arc-shaped surface of a top of the cup wall, the air pump inlet/outlet is arranged in the arc-shaped groove on one side of the protrusion and penetrates to an inner cavity of the total control upper housing.

8. The intelligent multifunctional cupping device according to claim 1, wherein the cup body can be a long-shaped cup, a chest cup, hip cup, or abdominal cup.

9. The intelligent multifunctional cupping device according to claim 1, wherein connection magnets II are respectively arranged at both ends of the connection groove, and connection magnets I corresponding to the connection magnets II in position are provided on both sides of a bottom end of the total control upper housing, the connection magnets II and the connection magnets I attract each other.

10. The intelligent multifunctional cupping device according to claim 1, wherein a top surface of the total control upper housing is respectively provided with a digital display screen for device function intensity, a power indicator light, and function control buttons, which are respectively connected with the control circuit board in the total control upper housing through circuits.
